(19) United States
(12) Patent Application Publication
(10) Pub. No.: US 2023/0330122 A1
(43) Pub. Date: Oct. 19, 2023

(54) PHARMACOLOGICAL TREATMENT OF AUTISM SPECTRUM DISORDER

(71) Applicant: Catarina Isabel Lemos Rodrigues de Sousa Cunha, Brooklyn, NY (US)

(72) Inventor: Catarina Isabel Lemos Rodrigues de Sousa Cunha, Brooklyn, NY (US)

(21) Appl. No.: 17/659,558

(22) Filed: Apr. 18, 2022

(51) Int. Cl. A61K 31/706; A61P 25/00

(52) U.S. Cl. CPC A61K 31/706; A61P 25/00

(57) ABSTRACT

A therapeutic method of treating symptoms of autism includes determining and preparing a dosage of nicotinamide mononucleotide effective to reduce compulsive behavior in the patient for a predetermined period and administering the dosage to the patient. NMN improves ASD symptoms by targeting inflammation in brain cells and enhancing mitochondrial bioenergetics, treating the root of the problem.

### BACKGROUND OF THE INVENTION

The present invention relates to autism spectrum disorder and, more particularly, to a pharmacological treatment therefor.

Autism Spectrum Disorder (ASD) comprises neurodevelopmental disorders characterized by impaired social communication and repetitive or restricted interests and behaviors. There is currently no specific drug or drug combination for autism that improves the real underlying problems, either to alter the physical condition of the brain or to substantially reduce the symptoms associated with autism. They only dampen the symptoms. Current drug treatments for autism have many side effects that negatively impact patients long-term.

One of the most life prevalent and challenging symptoms in autism are compulsions. Brain imaging studies have reported that certain regions of the brain are overactive in patients with obsessive compulsive disorder (OCD). Overactive brain regions are parts of the cortex (associated with decision making, reward and punishment, and error detection), the amygdala (associated with anxiety and fear), the thalamus (known as the “information hub”), and the dorsal striatum (associated with action selection and habit learning). The dorsal striatum promotes the selection of desirable actions and suppresses undesirable ones, a key impairment in patients with OCD. The dorsal striatum directly receives information from (and gives feedback to) every brain region implicated in OCD. A novel cancer drug, RG108 (DNA methyltransferase inhibitor) was shown to be successful in treating OCD in 3 different mouse models. However, cancer drugs are relatively harmful.

As can be seen, there is a need for an effective pharmacological treatment for the conditions underlying autism spectrum disorder.

### SUMMARY OF THE INVENTION

The present invention repurposes the supplement nicotinamide mononucleotide (NMN), the immediate biosynthetic precursor to nicotinamide adenine dinucleotide (NAD), to improve ASD symptoms.

In one aspect of the present invention, a therapeutic method of treating symptoms of autism is provided, comprising: determining an effective dosage of nicotinamide mononucleotide for a patient exhibiting symptoms of autism, wherein the effective dosage is effective to reduce compulsive behavior in the patient for a predetermined period; preparing the effective dosage of nicotinamide mononucleotide; and administering the effective dosage of nicotinamide mononucleotide to the patient.

These and other features, aspects and advantages of the present invention will become better understood with reference to the following drawings, description, and claims.

### BRIEF DESCRIPTION OF THE DRAWINGS

FIG. 1 is a diagram of nicotinamide mononucleotide structure;

FIG. 2 is a diagram of nicotinamide mononucleotide function;

FIG. 3 is a flow chart of a method of treating autism according to an embodiment of the present invention; and

FIG. 4 is a prior art diagram of brain circuitry;

FIG. 5 is a graph of experimental results of a method of treating autism according to an embodiment of the present invention; and

FIG. 6 is another graph of experimental results thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

The term “pharmaceutically acceptable carrier” is art-recognized and refers to a pharmaceutically acceptable material, composition, or vehicle, such as a liquid or solid filler, diluent, excipient, solvent, or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Subsequently, the term “pharmaceutically acceptable carrier” as used herein means any material or substance with which the active ingredient is formulated to facilitate its application or dissemination to the locus to be treated, for instance by dissolving, dispersing, or diffusing the composition, and/or to facilitate its storage, transport, or handling without impairing its effectiveness. The pharmaceutically acceptable carrier may be a solid, a liquid, or a gas which has been compressed to form a liquid, i.e., the compositions of this invention can suitably be used as concentrates, emulsions, solutions, granulates, dusts, sprays, aerosols, suspensions, ointments, creams, tablets, pellets, or powders. Further suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e., carriers and additives which do not create permanent damage to mammals. The pharmaceutical compositions of the present invention may be prepared in any known manner, for instance by homogeneously mixing, coating and/or grinding the active ingredients, in a one-step or multi-step procedure, with the selected carrier material and, where appropriate, other additives such as surface-active agents may also be prepared by micronization, for instance to obtain them in the form of microspheres usually having a diameter of about 1 to 1000 μm, namely for the manufacture of microcapsules for controlled or sustained release of the active ingredients.

Broadly, one embodiment of the present invention is a therapeutic method of treating autism symptoms by administering the supplement nicotinamide mononucleotide (NMN), the immediate biosynthetic precursor to nicotinamide adenine dinucleotide (NAD). NMN is a safe and effective novel treatment of autism.

Research has linked malfunctioning mitochondria and increased inflammation in brain cells to autism spectrum disorder. NMN has been shown to target inflammation and enhance mitochondrial bioenergetics, treating the root of the problem for autism which may improve ASD symptoms.

NMN is a harmless vitamin B3 with no recorded side effects to date. It is composed of a nicotinamide group, a ribose group, and a phosphate group. The enzyme responsible for making NMN in the body is called nicotinamide phosphoribosyltransferase (NAMPT). NAMPT attaches nicotinamide (a vitamin B3) to a sugar phosphate, 5-phosphoribosyl-1-pyrophosphate (PRPP). NMN can also be made from nicotinamide riboside (NR) through the addition of a phosphate group. Pharmacological activities of NMN include its role in cellular biochemical functions, cardioprotection, diabetes, Alzheimer's disease, and complications associated with obesity. The recent groundbreaking discovery of anti-aging (leading to demethylation) activities of this chemical moiety has added a valuable direction in the research involving this molecule.

The inventive method comprises administering a composition comprising NMN about twice a day to patients with autism. The NMN-containing composition may include a pharmaceutically acceptable carrier. The frequency of administration is not particularly limited and may be once a week, multiple times a week, once a day, twice a day, three times a day, etc.

The dosage may be optimized to achieve a specified outcome. The dosage for children may be primarily determined by age. For example, the dosage may range from about 100 mg to about 250 mg for children twice a day up to about 500 mg NMN twice a day for adults. For children, the dosage at 6 months-11 months may be about 100 mg twice a day; at 12-23 months may be about 150 mg twice a day; at 24 months-6-years may be about 200 mg twice per day; and at 6-12 years may be about 250 mg twice per day.

Any suitable means of administering the composition may be used. For example, administration may be selected from the group consisting of orally, topically, subdermally, and intravenously.

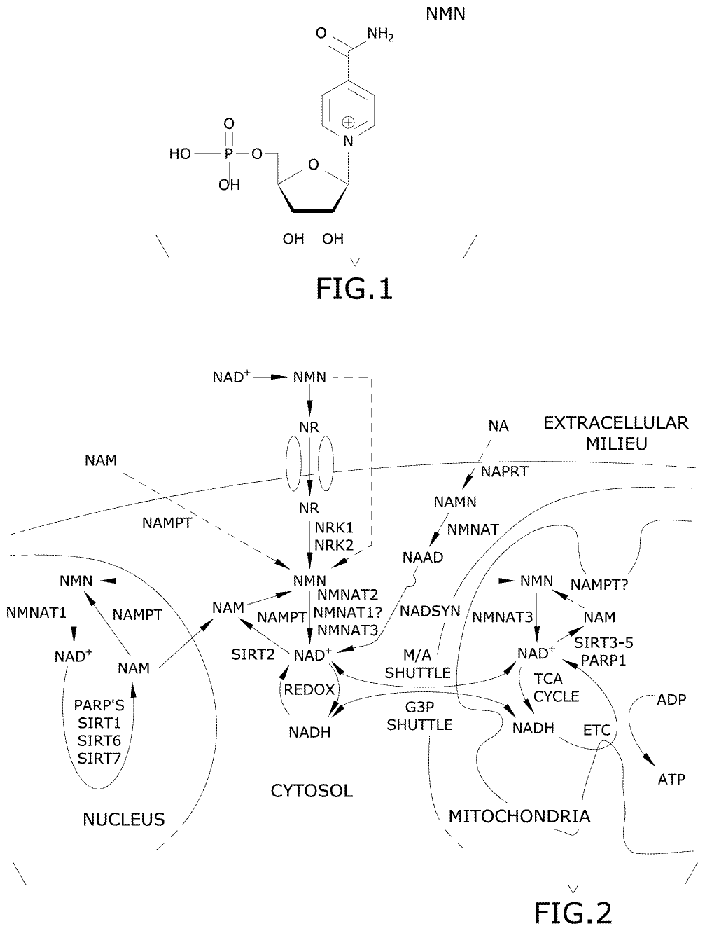

Referring to FIGS. 1 through 6, FIG. 1 illustrates the structure of the B3 vitamin supplement nicotinamide mononucleotide (NMN), having a nicotinamide group, a ribose group, and a phosphate group. NMN is a nucleotide that is most recognized for its role as an intermediate of nicotinamide adenine dinucleotide (NAD+) biosynthesis. NMN is a less harmful and cheaper alternative for treatment of OCD than cancer drugs.

NMN increases NAD+ levels and inhibits chronic inflammation by the pathways illustrated in FIG. 2. NAD+ has emerged as a critical component that maintains mitochondrial fitness.

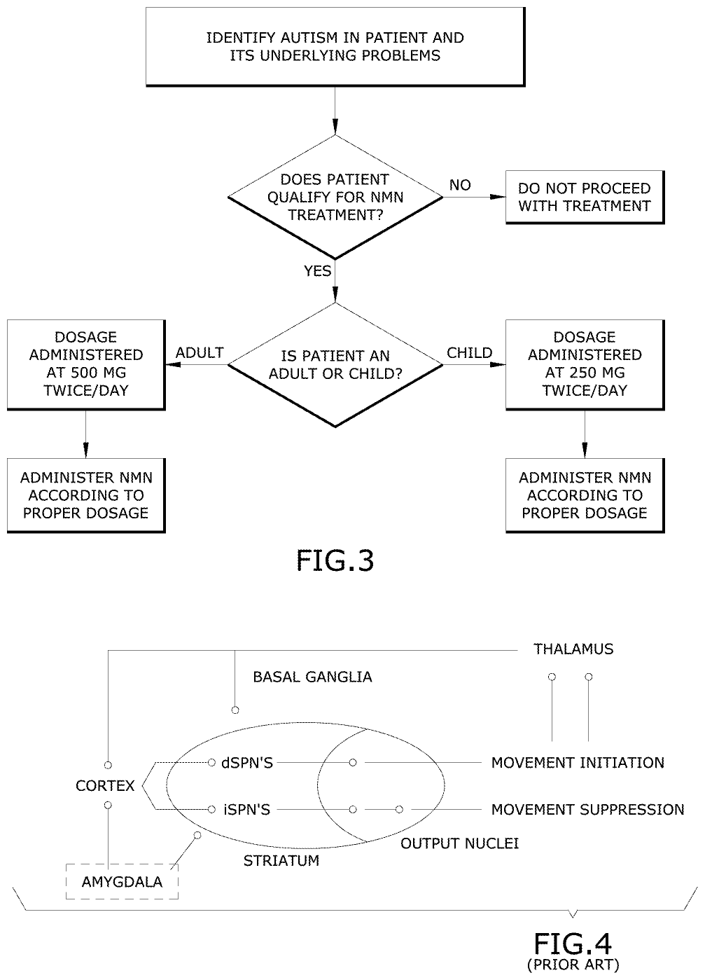

FIG. 3 is a flow diagram of a method of treating autism symptoms with NMN according to an embodiment of the present invention. Note that NMN is administered when indicated.

The precise brain circuitry underlying OCD is not known. FIG. 4 illustrates interactions within the brain which offer some clues. The basal ganglia is instrumental in selecting desirable actions and suppressing undesirable ones. Much of this occurs in the striatum, the major input nucleus of the basal ganglia. The striatum has been implicated as a mediator of OCD-like behaviors in both humans and animal models. See Burguiere et al., 2015.

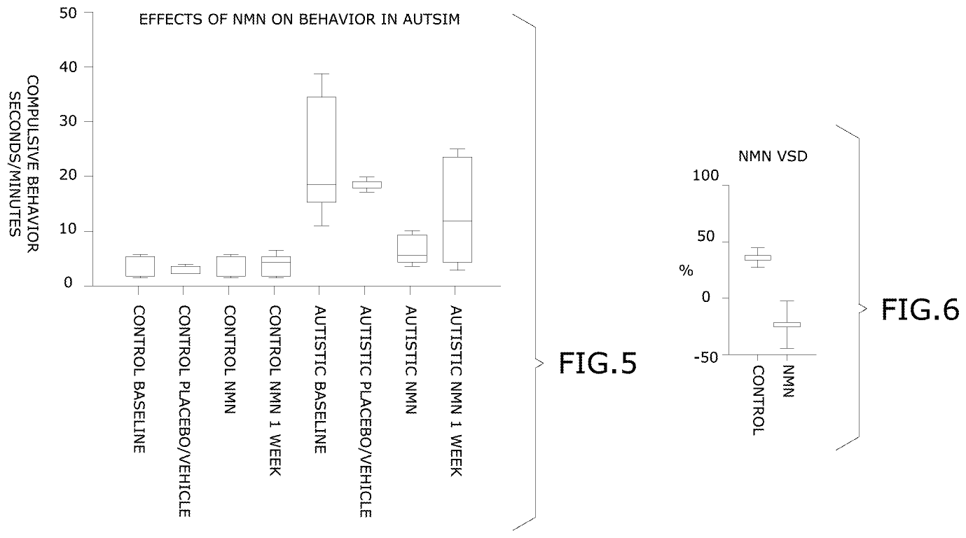

FIGS. 5 and 6 present data collected by experiments using a SH3 And Multiple Ankyrin Repeat Domains 3 (SHANK3) autism mouse model. SHANK3 was the first to be associated with autism which was used by Joao Peca et al. (2011) to create the mouse model currently in use. The SHANK3 mice exhibit self-injurious repetitive grooming and deficits in social interaction. The SHANK family of proteins has been strongly implicated as a contributing factor in autism in certain individuals and sits at the core of the alleged autistic pathway. The SHANK proteins lie at the heart of this synaptic map of autistic pathology, binding to Metabotropic Glutamate Receptors (mGluRs) indirectly, via the Homer family of adaptor proteins, and to both N-methyl-D-aspartate receptors (NMDARS) and Neuroligins (NLGNs) indirectly, via guanylate kinase-associated protein (GKAP) and Postsynaptic density protein 95 (PSD-95). They also link to the actin cytoskeleton, providing a multivalent scaffold upon which to build the postsynaptic density.

Compulsive behavior was used as a readout to evaluate the effectiveness of autistic symptom amelioration through NMN treatment. Behavioral results are shown in FIG. 5, which presents compulsive behavior in seconds observed over a 5-minute period. A control group and an autistic group were subject to specified treatment. A baseline was recorded for both groups. The effect of a placebo was evaluated for both groups. NMN was administered to subjects of each group and the results were recorded. The results of treatment with NMN over 1 week were also recorded. THE RESULTS SHOW THAT NMN IS A SAFE NOVEL TREATMENT AND PREVENTIVE DRUG FOR AUTISM, significantly reducing the exhibition of compulsive behavior.

FIG. 6 shows a percent change in neuroimaging neural activity in the striatum with voltage sensitive dyes (VSD) from NMN treatment of control wild-type (WT) mice to NMN treatment of the SHANK mice. A voltage sensitive dye, Tetramethylrhodamine, ethyl ester (TMRE) was designed and synthesized in a lab to measure absolute membrane potential via a Nernstian redistribution mechanism. Permeable highly fluorescent cations distribute across the membrane according to the Nernst equation. It is also possible to measure changes in membrane potential as a result of signaling activity.

It should be understood, of course, that the foregoing relates to exemplary embodiments of the invention and that modifications may be made without departing from the spirit and scope of the invention as set forth in the following claims.

What is claimed is:

1. A therapeutic method of treating symptoms of autism, comprising:
determining an effective dosage of nicotinamide mononucleotide for a patient exhibiting symptoms of autism, wherein the effective dosage is effective to reduce compulsive behavior in the patient for a predetermined period;
preparing the effective dosage of nicotinamide mononucleotide; and
administering the effective dosage of nicotinamide mononucleotide to the patient.

2. The therapeutic method of claim 1, wherein the effective dosage is administered once per day to three times per day.

3. The therapeutic method of claim 1, wherein the effective dosage is determined by age of the patient and ranges from about 100 mg to about 500 mg when administered twice per day.

4. The therapeutic method of claim 1, wherein the effective dosage of nicotinamide mononucleotide is provided as a composition including a pharmaceutically acceptable carrier.

5. The therapeutic method of claim 1, wherein the step of administering is selected from the group consisting of orally, topically, subdermally, and intravenously.
